(19) United States
(12) Patent Application Publication
(10) Pub. No.: US 2016/0166572 A1
(43) Pub. Date: Jun. 16, 2016

(54) BENEFICIAL EFFECTS OF Lp-PLA2 INHIBITORS IN TREATMENT OF DIABETIC RETINOPATHY AND AGE-RELATED MACULAR DEGENERATION

(71) Applicant: Rowan University, Glassboro, NJ (US)

(72) Inventor: Robert G. Nagele, Turnersville, NJ (US)

(73) Assignee: Rowan University, Glassboro, NJ (US)

(21) Appl. No.: 14/965,479

(22) Filed: Dec. 10, 2015

Related U.S. Application Data
(60) Provisional application No. 62/204,209, filed on Aug. 12, 2015.
(60) Provisional application No. 62/090,328, filed on Dec. 10, 2014.

(51) Int. Cl. A61K 31/517; A61B 5/00; A61B 3/10; A61K 31/4709; A61K 45/06

(52) U.S. Cl. CPC A61K 31/517; A61K 31/4709; A61K 45/06; A61B 3/1005; A61B 3/102; A61B 5/4082; A61B 5/4088; A61B 5/4848

(57) ABSTRACT

The present invention relates to methods of detecting, diagnosing and treating diseases and disorders associated with increased BBB and BRB permeability.

### CROSS-REFERENCE TO RELATED APPLICATIONS

This application claims the benefit of U.S. Provisional Application Ser. Nos. 62/090,328, filed Dec. 10, 2014 and 62/204,209, filed Aug. 12, 2015, which are hereby incorporated by reference in its entirety.

### FIELD OF THE INVENTION

The present invention relates generally to methods for the detection, treatment and/or prevention of age-related eye diseases and disorders, and more particularly to the detection, treatment and/or prevention of these diseases and disorders associated with abnormal blood-retinal barrier (BRB) permeability using agents that inhibit the expression and/or activity of the Lp-PLA2 protein.

### BACKGROUND

The retina has a functional blood-tissue barrier referred to as the blood-retinal barrier (BRB). This barrier consists of inner and outer portions, each with distinct ontogenetic origins and functional capacities. The present application has focuses only on the inner BRB, and thus our use of the term BRB refers only to the inner aspect of the BRB. This part of the BRB is composed of a monolayer of vascular endothelial cells tasked with restricting the movement of plasma components from the blood into the immune-privileged retina, and closely resembles the BBB of the brain in both structure and function. In patients with diabetes mellitus (DM), chronic extravasation of plasma components via a compromised BRB has been widely reported and implicated in the pathogenesis of diabetic retinopathy. Diabetic retinopathy is one of the early onset, long-term complications associated with DM and is regarded as the primary cause of partial or complete vision loss in these patients. Details of the pathogenesis of diabetic retinopathy in patients with chronic DM remain elusive.

### SUMMARY OF THE INVENTION

In the study of an animal model of DM and hypercholesterolemia (HC) and the associated effect on BBB permeability, it has been observed that an increased BBB permeability is morphologically evident as perivascular leak clouds and coincident with the selective binding of immunoglobulin G (IgG) to pyramidal neurons in the cerebral cortex as well as heightened intracellular deposition of the amyloid beta₁₋₄₂peptide (Aβ42) in these same neurons. Treatment with lipoprotein-associated phospholipase A2 inhibitors such as Darapladib, is able to reverse BBB permeability as well as IgG binding and Aβ42 deposition in cortical pyramidal neurons. In particular, alleviated changes are observed in retinal layer thickness and architecture, thus providing a novel approach for the early detection, diagnosis, treatment and prevention of various eye diseases associated with age-related macular degeneration (AMD), diabetic retinopathy (DR).

Moreover, changes in the thickness of retinal layers are accompanied by altered expression of key tight junction proteins. Claudin5 and occludin in the cerebral cortex of the brain regions in disease-animal models have been found to be over-expressed. The increased expression of these key tight junction proteins may be a part of a compensatory mechanism implemented in response to induced BBB breach. Therefore, BRB integrity can be used to gauge the overall health of BBB.

An aspect of the invention provides a method of treating or preventing an eye disorder or disease in a subject. The method includes the steps: (a) identifying a subject with an eye disease or disorder or at risk of having the eye disease or disorder by measuring changes in the thickness of one or more retinal layers, including the ganglion cell layer (GCL), outer nuclear layer (ONL), and inner-segment (IS); and (b) administering to the subject in need thereof a pharmaceutical composition comprising an effective amount of an agent for inhibiting the activity or expression of the Lp-PLA2 protein. The eye disorder or disease is characterized by one or more disease states selected from vitreous hemorrhage, progressive fibrovascular proliferation, retinal detachment, rubeosis iridis, and neovascular glaucoma. The measurement of the thickness of the retinal layers or sublayers can be compared with a reference such as the thickness of a normal retinal layer. In some embodiments, the eye disorder or disease is selected from central retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema. In some embodiments, the eye disorder or disease is associated with age-related macular degeneration (AMD), diabetic retinopathy (DR), or both.

In some embodiments, the agent is darapladib or rilapladib. In some embodiments, the method further includes administering to the subject a second agent selected from Corticosteroids, VEGF inhibitors, PKC inhibitors, and growth hormone inhibitors. In some embodiments, the method further includes measuring one or more retinal layers after the initial administration of the agent and adjusting the dosage of the agent accordingly. In some embodiments, the thickness is measured with optical coherence tomography.

Another aspect of the invention provides a method of determining whether a subject has, or is at risk of having, an eye disease or disorder. The method includes the steps: (a) measuring the thickness of one or more retinal layers selected from ganglion cell layer (GCL), outer nuclear layer (ONL), and inner-segment (IS); (b) comparing the measured thickness with a reference such as the thickness of a normal retinal layer; and (c) generating a report specifying that whether the subject has, or is at risk of having, the eye disease or disorder.

Another aspect of the invention provides a method of detecting the extent of a neurodegenerative disease or the risk of the neurodegenerative disease in a subject comprising measuring the thickness of one or more retinal layers selected from ganglion cell layer (GCL), outer nuclear layer (ONL), and inner-segment (IS).

In some embodiments, the method further includes comparing the measured thickness with a reference; and generating a report specifying that the extent of the neurodegenerative disease or the risk of the neurodegenerative disease in the subject. In some embodiments, the neurodegenerative disease is selected from Alzheimer's disease, Multiple Sclerosis, Parkinson's disease, frontotemporal dementia, vascular cognitive impairment (formerly vascular dementia), and dementia with Lewy body.

Another aspect of the invention provides a method of monitoring the treatment response in a subject having a neurodegenerative disease or an eye disease or disorder. The method includes the step of measuring the thickness of one or more retinal layers selected from ganglion cell layer (GCL), outer nuclear layer (ONL), and inner-segment (IS) after administration of an agent to the subject. In some embodiments, the method further includes comparing the thickness of the one or more retinal layers with a reference and generating a report, wherein the reference is the thickness of the one or more retinal layers prior to administration of the agent.

### BRIEF DESCRIPTION OF THE DRAWINGS

FIGS. 1A, 1B and 1C illustrate the effect of DMHC induction and Darapladib treatment on the porcine retina (Ret.).

FIGS. 2A, 2B, 2C, 2D, 2E and 2F illustrate the measurement of relative thickness of retinal layers.

FIGS. 3A, 3B, 3C, 3D and 3E show that DMHC induced lowering of cell count in GCL and INL was reversed by Darapladib treatment.

FIGS. 4A, 4B, 4C and 4D show that DMHC pigs have the highest incidence of IgG labeled ganglion cells.

FIGS. 5A, 5B, 5C and 5D show that DMHC induction and Darapladib treatment modify GFAP expression.

FIGS. 6A, 6B, 6C, 6D, 6E, 6F and 6G show that BVECs of cerebral cortex demonstrate selective expression of claudin 5 and occludin in all treatment groups.

FIGS. 7A, 7B, 7C, 7D, 7E and 7F show that DMHC induction and Darapladib treatment influence the localization of claudin 5 and occludin in cortical vasculature.

FIGS. 8A and 8B show that DMHC induction and Darapladib treatment modify expression of the tight junction proteins, claudin5 and occluding, and GFAP.

### DETAILED DESCRIPTION

The present invention identified degeneration and edematous changes in certain specific retinal layers that are known to negatively affect vision when a diseased state such as DMHC was induced. Importantly, the marked changes relating to the diseased state in specific retinal layer thicknesses and nucleus density are clearly reversed when a Lp-PLA₂ inhibitor is administered to a subject in need. Meanwhile, a subsequent reversion to control levels of GFAP positivity suggests that the need for Muller cells to aid in regenerative and protective roles decreases with the decreasing levels of vascular pathology. This is an unexpected observation, especially when comparing with the corresponding Aβ₁₋₄₂ accumulation and how it relates to Alzheimer's disease (AD).

Patients with eye diseases or disorders at various stages or at risk of developing eye diseases may have changes in thickness at one or more retinal layers including ganglion cell layer (GCL), outer nuclear layer (ONL), and inner-segment (IS)/outer-segment (OS) layer. In general, increased BRB permeability is associated with reduction in thickness for layers of GCL, ONL and IS/OS. Meanwhile, the abnormal BRB permeability also leads to increase in thickness for the inner plexiform layer (IPL) as shown in DMHC animals.

Throughout this patent document, various publications are referenced. The disclosures of these publications in their entireties are hereby incorporated by reference into this application in order to more fully describe the state of the art to which the disclosed matter pertains. While the following text may reference or exemplify specific steps of a method of detection, diagnosis, or treatment, it is not intended to limit the scope of the invention to such particular reference or examples. Various modifications may be made by those skilled in the art, in view of practical and economic considerations, such as the instrument of measuring the retinal layers and the amount of the agent.

As used herein, the articles “a” and “an” as used herein refers to “one or more” or “at least one,” unless otherwise indicated. That is, reference to any element or component of the present invention by the indefinite article “a” or “an” does not exclude the possibility that more than one of the element or component is present.

As used herein, the term “about” refers to the referenced numeric indication plus or minus 10% of that referenced numeric indication.

As used herein, the term “subject” is intended to include human and non-human animals. Non-human animals includes all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include human patients in need of detection, diagnosis or treatment of an eye disease or a neurodegenerative disease.

As used herein, the term “disease” or “disorder” is used interchangeably herein, and refers to any alteration in state of the body or of some of the organs, interrupting or disturbing the performance of the functions and/or causing symptoms such as discomfort, dysfunction, distress, or even death to the person afflicted or those in contact with a person. A disease or disorder can also relate to a distemper, ailing, ailment, malady, disorder, sickness, illness, complaint or affectation.

As used herein, the term “BBB permeability” is commonly referred to by persons in the art as “leaky BBB”. The terms are used interchangeably herein to refer to impaired BBB integrity and increased vascular permeability. For example, a permeable BBB allows transit of molecules through the BBB that an intact BBB would normally exclude from the brain tissue, for example, Ig molecules, complement proteins, serum albumin and numerous other proteins. An assay to determine the presence of a permeable BBB can be, for example, to assess the presence of extravascular Ig in the brain tissue which is normally restricted to the lumen of blood vessels when the BBB is functioning normally (i.e., when the BBB is not permeable).

As used herein, the term “BRB permeability” is commonly referred to by persons in the art as “leaky BRB” and refers to impaired BRB integrity and increased vascular permeability. For example, a permeable BRB allows transit of molecules through the BRB that an intact BRB would normally exclude from the retinal tissue, for example, Ig molecules, complement proteins, serum albumin and numerous other proteins. An assay to determine the presence of a permeable BRB can be, for example, to assess the presence of extravascular Ig in the retinal tissue which is normally restricted to the lumen of blood vessels when the BRB is functioning normally (i.e., when the BRB is not permeable).

As used herein, the term “effective amount” refers to the amount of therapeutic agent of pharmaceutical composition to reduce, stop, alleviate or prevent at least one symptom of the eye diseases or disorders disclosed herein. For example, an effective amount for treating diabetic macular edema would include an amount sufficient to improve vision, usually achieved by reducing the amount of macular edema caused by leakage from intraretinal capillaries. The amount of edema can be estimated by the amount of retinal thickening detected by a noninvasive technique such as OCT and/or by the leakage detected on fluorescein angiography. An effective amount as used herein would also include an amount sufficient to prevent or delay the development of macula edema and associated vision loss. An effective amount as used herein would also include an amount sufficient to prevent or delay the development of a symptom of the disease, alter the course of a symptom of the disease (for example but not limited to, slow the progression of a symptom of the disease), or reverse a symptom of the disease.

As used herein, the term “Lp-PLA2” refers to the protein target inhibited by the methods as disclosed herein. Lp-PLA2 is used interchangeably with lipoprotein associated phospholipase A2, also previously known in the art as Platelet Activating Factor Acetyl Hydrolase (PAF acetyl hydrolase). Human Lp-PLA2 encoded by nucleic acid and the corresponding sequence IDs are disclosed in U.S. Pat. No. 5,981,252, which is specifically incorporated herein in its entirety by reference.

As used herein, the term “prevent” refers to detecting the risk or early signs/symptoms of a disease or disorder and reducing or eliminating such risk or symptoms prophylactically.

As used herein, the term “treating” includes reducing, alleviating at least one adverse effect or symptom of a condition, disease or disorder associated with the eye diseases and disorders described herein. Methods for measuring positive outcomes of treatment include, but are not limited to reduction or maintenance of sub-retinal edema, measured by OCT, reduction in the loss or maintenance of vision, or the gain of vision as assessed by best corrected visual acuity.

It is discovered that Lp-PLA₂ inhibitors such as Darapladib are able to reverse the effects of DMHC on the thickness of the GCL, ONL and IS/OS layers. Darapladib can also reduce IPL thickness in DMHC animals to near control levels. In terms of the impact on cells, results have shown that DMHC is associated with significant reduction in the numbers of nuclei and therefore cells within the GCL and INL compared to controls. On the other hand, treatment with Darapladib inhibited cell loss within these two retinal layers such that measured cell numbers are comparable to controls.

With regard to the level of IgG, increased presence of IgG-positive ganglion cells in the retina is detected in the retina of DMHC animal models. Again, Lp-PLA₂ inhibitors such as Darapladib are able to normalize the level of IgG in the retina. GFAP expression can also be increased in the DMHC retina compared to controls and Darapladib-treated animals.

Further, the present invention establishes a clear correlation between BRB and BBB, and sets the stage for methods of treating patients suffering from disruption of BRB associated with any pathophysiology. With the ability to utilize current ophthalmological techniques to view the BRB for its leaky areas and extent of leakiness, it is now feasible to use such factors as a surrogate when evaluating BBB pathologies and diagnosing eventual neurodegenerative disease states founded in such vascular demise.

Although claudin5 and occludin and their relationship to the BBB have been extensively studied, their individual contributions to the functional integrity of the BBB remain unresolved until the present invention. The correlation between increased BBB permeability and claudin5 and occludin expression during DM and HC states has been investigated.

The present invention revealed quantitative changes in the expression of the tight junction proteins, claudin 5 and occludin, at the gene and protein levels in the cerebral cortex of DMHC pigs. Claudin5 and occludin are expressed in BVECs that serve as key structural and functional components of the BRB and BBB. A pig cerebral cortex model has been chosen over the highly laminated retina for this purpose because of the much greater volume of cerebral cortex tissue available for analysis and the fact that the greater number and more randomized distribution of blood vessels in the cortex makes this brain region more amenable to quantification based on protein localization. In the anterior and middle cerebral cortex, DMHC-only pigs showed the greatest expression of claudin5, followed by Darapladib-treated DMHC and control pigs. The DMHC-Darapladib group exhibited less claudin5 expression than the DMHC group, but the differences between these two groups did not reach statistical significance. Interestingly, treatment of DMHC animals with Darapladib resulted in a reduced expression of claudin5 to levels similar to controls. In the posterior cortex, the DMHC-Darapladib group showed the highest expression of claudin5, followed by DMHC and controls, with differences between all treatment groups attaining statistical significance. In contrast to claudin5, expression of occludin varied from one brain region to another and failed to show any reproducible trend. This failure to detect any trend in occludin localization and expression may be, in part, explained by its apparently limited role in maintaining BBB integrity. The strongest evidence diminishing a role for occludin in maintaining BBB integrity comes from occludin knockout mouse studies. Occludin knockout mice are not only viable, but an ultrastructural study also revealed the presence of normal tight junctions both in the CNS vascular endothelial cells and intestinal epithelial cells.

From these data, chronic DMHC and treatment of DMHC animals with Darapladib had a clear impact on claudin5 expression. Without being bound to any particular theory, it is suggest that the overall increased expression of tight junction proteins could be a part of a compensatory mechanism implemented by BVECs in response to DMHC-induced BBB breach. Further, a similar increase in BRB permeability in the retinas of the same DMHC animals as well as a curtailment of BRB compromise in DMHC animals treated with Darapladib was observed. Therefore, the similar actions and responses between the BRB of the retina and BBB in the cerebral cortex indicate a good correlation between the functional status of these barriers. This observation lays the foundation for detection of BRB compromise in the eye as predictive factors of a similar compromise of the BBB in the brain and thus presents broad diagnostic utility.

Accordingly, an aspect of the present invention provides a method of treating or preventing an eye disorder or disease in a subject by administering to the subject in need thereof a pharmaceutical composition containing an agent for inhibiting the activity or expression of the Lp-PLA₂ protein.

In order to determine a suitable agent and the appropriate amount of administration, the method may also contain a step of detecting or identifying the disease condition or risk of disease condition. The detecting step includes measuring the thickness of one or more retinal layers selected from ganglion cell layer (GCL), outer nuclear layer (ONL), and inner-segment (IS). In some embodiments, the thickness of the thickness of GCL, ONL, or both of these layers is measured. In some embodiments, the method further involves measuring the thickness of the inner plexiform layer (IPL). Determination of a disease or risk of a disease can be made by comparing the measured thickness with a reference. The reference may be the baseline thickness of a retinal layer prior to administration of the agent. Alternatively, the reference is the normal thickness of a retinal layer in a healthy subject. A reference may also be a mean or median thickness in a population group of interest, particularly suffering from the same pathological condition.

Various procedures such as fluorescein angiograms can be used to assess functional integrity of the BRB. Exemplary approaches for measuring retinal layers are disclosed in U.S. Patent Application No. 20140218686, U.S. Patent Application No. 20080309881, and U.S. Pat. No. 8,801,187, the entire disclosure of which is hereby incorporated by reference.

Because of the ease and greater predictability, the method of the present invention can be applied to the early detection and diagnosis of various eye disease or disorder characterized by vitreous hemorrhage, progressive fibrovascular proliferation, retinal detachment, rubeosis iridis, and neovascular glaucoma. Exemplary diseases or disorders include eye disorder or disease is selected from central retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema. In some embodiments, the eye disorder or disease is associated with age-related macular degeneration (AMD), diabetic retinopathy (DR), or both.

Various agents are suitable for inhibiting the activity or expression of the Lp-PLA₂ protein. Inhibitors of Lp-PLA2 can be a chemical, small molecule, large molecule or entity or moiety, including without limitation synthetic and naturally-occurring non-proteinaceous entities. In certain embodiments the agent is a small molecule having the chemical moieties as disclosed herein. Exemplary compounds include those disclosed in U.S. Patent Application No. 20130267544, the entire disclosure of which is hereby incorporated by reference. In some embodiments, the agent is Darapladib, rilapladib or any agent or condition that influences the activity or expression of Lp-PLA2.

In at least one embodiment, the present invention is directed to a method for evaluating a compound that may be effective for treatment of an eye disorder or disease, comprising: (a) contacting a test compound with a cell overexpressed with GFAP; and (b) detecting the change in the expression of GFAP in the cell. In yet another embodiment, the eye disorder or disease is characterized by one or more disease states selected from vitreous hemorrhage, progressive fibrovascular proliferation, retinal detachment, rubeosis iridis, and neovascular glaucoma. The change in the expression of GFAP pre and post contacting of the test compound with the overexpressed cells can be done by variety of methodologies known to those of ordinary skill in the art.

In some embodiments, a secondary agent may be used in conjunction with the above mentioned Lp-PLA2 inhibitors. Examples of such secondary agents include Corticosteroids, VEGF inhibitors, PKC inhibitors, and growth hormone inhibitors. The secondary agent may be administered separately or simultaneously with a Lp-PLA2 inhibitor.

Another aspect of the invention provides a method for determining whether a subject has, or is at risk of having, an eye disease or disorder. The method generally includes measuring the thickness of one or more retinal layers selected from ganglion cell layer (GCL), outer nuclear layer (ONL), and inner-segment (IS); comparing the measured thickness with a reference; and generating a report specifying that whether the subject has, or is at risk of having, the eye disease or disorder. The reference may be the thickness of a retinal layer prior to administration of the agent. Alternatively, the reference is the normal thickness of a retinal layer in a healthy subject. A reference may also be a mean or median thickness in a population group of interest.

In some embodiments, the eye disorder or disease is characterized by one or more disease states including for example vitreous hemorrhage, progressive fibrovascular proliferation, retinal detachment, rubeosis iridis, and neovascular glaucoma. The eye disorder or disease that can be detected by the present method includes for example retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema. In some embodiments, the eye disorder or disease is associated with age-related macular degeneration (AMD), diabetic retinopathy (DR), or both.

Another aspect of the invention provides a method of detecting the extent of a neurodegenerative disease or the risk of the neurodegenerative disease in a subject. The method involves measuring the thickness of one or more retinal layers of ganglion cell layer (GCL), outer nuclear layer (ONL), and inner-segment (IS). The method may also include the steps of comparing the measured thickness with a reference; and generating a report specifying that the extent of the neurodegenerative disease or the risk of the neurodegenerative disease in the subject. The reference may be the thickness of a retinal layer prior to administration of the agent. Alternatively, the reference is the normal thickness of a retinal layer in a healthy subject. A reference may also be a mean or median thickness in a population group of interest.

As described herein, BRB breakdown can be detected years ahead of clinical diagnosis of diseases such as Diabetic retinopathy. The present invention further establishes that BBB and BRB are functionally similar and BRB integrity can be used to gauge the overall health of BBB. Thus, detecting any aberrations in BRB helps in pre-symptomatic diagnosis of various neurodegenerative diseases.

An increased expression of claudin5 in BVECs has been observed in DMHC animal models, thus corroborating its role in maintaining BBB integrity in cerebral cortex. Increased expression of claudin5 in cortical vasculature of DMHC pigs may be part of a damage-response mechanism elicited by BVECs in-response to challenges to BBB functional integrity. If true, then the pursuit of therapeutic strategies aimed at increasing claudin5 expression with the intent of fortifying BBB and BRB integrity may have merit. Similarly, it is observed that chronic DMHC leads to significant reductions in the thicknesses of some retinal layers as a result of cell loss, most notably in the retinal ganglion cells that also exhibited the highest level of IgG labeling. As in the IgG-mediated degenerative changes reported in the cerebral cortex of these animals, it is proposed that chronic IgG binding to ganglion cells may lead to their death and the observed loss of these cells from ganglion cell layer.

Accordingly, measuring the thickness of retinal layers such as GCL, ONL and IS provides an early detection for the risk and diagnosis of neurodegenerative diseases. The method of the present invention can be applied to various neurodegenerative diseases including for example any form of optic neuritis, glaucoma, HIV, AIDS dementia complex, adrenoleukodystrophy, Alexander disease, Alpers' disease, Alzheimer's disease, amyotrophic lateral sclerosis, ataxia telangiectasia, Batten disease, bovine spongiform encephalopathy, Canavan disease, cerebrovascular pathology, Charcot-Marie-Tooth disease, corticobasal degeneration, Creutzfeldt-Jakob disease, dementia, dementia with Lewy bodies, diabetic neuropathy, diffuse myelinoclastic sclerosis, fatal familial insomnia, frontotemporal lobar degeneration, giant axonal neuropathy, glaucoma, Huntington's disease, Kennedy's disease, Krabbe disease, leprosy, Lyme disease, Machado-Joseph disease, malaria, multiple sclerosis, multiple system atrophy, neuroacanthocytosis, neuropathy, Niemann-Pick disease, neurofilament inclusion body dementia, neuromyelitis optica, Parkinson's disease, variants of classical Parkinson's disease, Pick's disease, primary lateral sclerosis, progressive supranuclear palsy, Refsum's disease, Sandhoff disease, spinocerebellar ataxia, stroke, subacute combined degeneration of spinal cord, Tabes dorsalis, Tay-Sachs disease, toxic encephalopathy, transmissible spongiform encephalopathy, traumatic brain injury any toxic encephalopathy, wobbly hedgehog syndrome and vasculitis.

In some embodiments, the present invention can be applied to the detection, treatment, and/or prevention of neurodegenerative diseases including for example, Alzheimer's disease, Multiple Sclerosis, Parkinson's disease frontotemporal dementia, vascular cognitive impairment (formerly vascular dementia), and dementia with Lewy body.

The method of detection may be used in conjunction with other known methods for the detection and diagnosis of neurodegenerative diseases. Recently reported diagnosis methods include for example those disclosed in U.S. Patent Application No. 20140038834, U.S. Pat. No. 7,892,751, and U.S. Patent Application No. 20120094295, the entire disclosure of which is incorporated by reference.

Another aspect of the invention provides a method of monitoring the treatment response in a subject having a neurodegenerative disease or an eye disease or disorder. The method includes measuring the thickness of one or more retinal layers of ganglion cell layer (GCL), outer nuclear layer (ONL), and inner-segment (IS) after administration of an agent to the subject. By comparing the thickness of a retinal layer at various stages of the treatment regimen, the effectiveness of the treatment can be conveniently monitored.

In some embodiments, the method includes comparing, at various stages, the thickness of a retinal layer with a reference. The reference may be the thickness of a retinal layer prior to administration of the agent. Alternatively, the reference is the normal thickness of a retinal layer in a healthy subject. A reference may also be a mean or median thickness in a population group of interest.

In some embodiments, the disease to be monitored is a neurodegenerative disease described herein. In some embodiments, the disease is an eye disease or disorder described herein.

Another aspect of the invention provides a system including a detection unit and a subject population database unit. The system enables the measurement of a retinal layer as well as a rapid determination of risk and extent of various eye diseases and neurodegenerative diseases described herein. The detection unit provides thickness measurements of one or more retinal layers described herein. For example, the detection unit can be configured to utilize optical coherence tomography in the measurement of retinal layers. The database unit allows for comparison of the measured thickness with a reference, which may be the thickness of a normal retinal layer or the thickness of a diseased or pre-diseased retinal layer. The thickness reference for a normal retinal layer or a diseased or pre-diseased retinal layer may be an actual measurement or a calculated one based on statistics obtained from subject population. For example, by studying the relevant subject population, it can be determined that an increase in the thickness of in a retinal layer may be associated a risk of about 10%, 20%, 30%, 40%, 50%, 60%, 70%, 80% or more to develop an eye disease or a neurodegenerative disease.

The composition of the present invention may be prepared by mixing the Lp-PLA₂ inhibitor having the desired degree of purity with optional physiologically acceptable carriers, excipients or stabilizers (Remington's Pharmaceutical Sciences 16th edition, Osol, A. Ed. (1980)). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS, or polyethylene glycol (PEG).

As described above, Lp-PLA₂ inhibitors are useful for the prevention, treatment, control, amelioration, or reduction of risk of the diseases, disorders and conditions associated with increased permeability of BBB and BRB. The dosage of the agent as an active ingredient in the compositions of this invention may be varied so that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.001 to 100 mg/kg. of body weight daily are administered to the patient, e.g., humans and elderly humans. The dosage range will generally be about 0.5 mg to 10 g per patient per day which may be administered in single or multiple doses.

The composition of the present invention may be administered by oral, parenteral (e.g., intramuscular, intraperitoneal, intravenous, ICY, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. For example, a therapeutic agent can be administered orally or locally. Examples of local administration include topical ophthalmic administration. The terms “administration of” and or “administering a” agent or composition should be understood to mean providing a Lp-PLA2 inhibitor or a composition thereof to the individual in need of treatment.

As mentioned above, it is understood that Lp-PLA2 inhibitors can be administered at prophylactically effective dosage levels to prevent the above-recited conditions and disorders, as well as to prevent other conditions and disorders associated with increased BBB or BRB permeability.

### EXAMPLES

### Materials and Methods:

Porcine model, tissue handling and processing

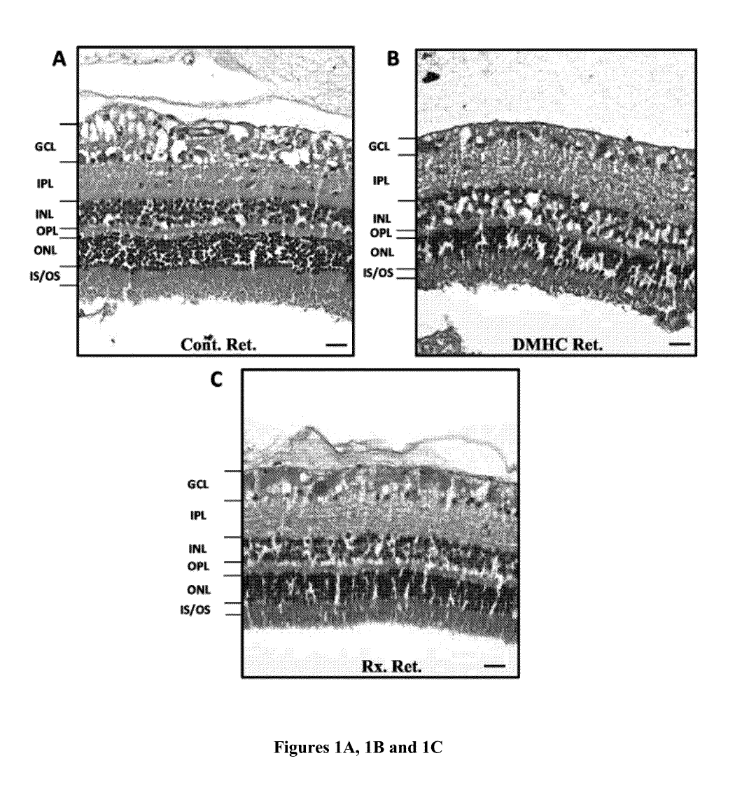

Briefly, after induction of diabetes by a single injection of Streptozotocin, animals were fed a high cholesterol diet. After a month of DMHC induction and acclimation, animals were randomly segregated to untreated DMHC and Darapladib-treated DMHC groups. Darapladib-treated pigs received 10 mg/kg/d of Darapladib orally. Three of the pigs were spared from DMHC induction and served as age-matched, non-DMHC normal controls. Twenty four weeks after acclimation, animals were euthanized and their brains and eyes were immediately removed. Brains were divided anteroposteriorly into anterior, middle and posterior regions. For each of these brain regions, 2-3 tissue blocks were generated for routine tissue fixation (10% formalin) or freezing and histological processing. The anterior cortical region comprised of anterior ⅔ of frontal lobe and anterior ⅓ of temporal lobe. The middle cortical region contained mid ⅓ of temporal lobe and anterior ½ of the parietal lobe. The posterior cortex included the posterior ½ of the parietal lobe, posterior ⅓ of the temporal lobe and the occipital lobe. Eyes were dissected longitudinally to sever the retina at the optic nerve. The sections were fixed in 10% formalin and embedded as flattened disc in paraffin. The tissue samples were processed using routine histology techniques. Standard Hematoxylin & Eosin (H&E) staining (FIG. 1) was carried out in retinal sections.

### Determination of Retinal Thickness

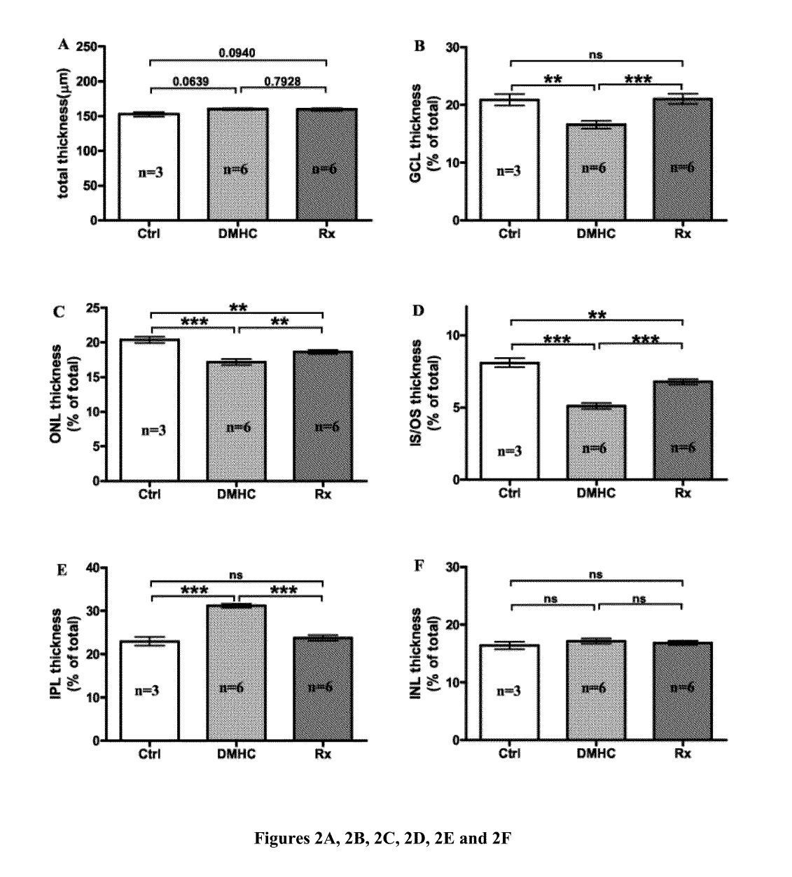

The remarkably uniform and laminated histological architecture of the retina lends itself well to direct measurements of the thickness of the various layers as shown in FIG. 1. Using the image analysis software, ImagePro 7.0 (Media Cybernetics, Silver Spring, Md.), the thickness of all retinal layers were measured. The thickness of each retinal layer was measured in three separate retinal sections obtained from each animal. In each section, measurements were made at two randomly selected locations in the middle part of retina. Results were averaged for each pig. The identity of specimens was blinded and two independent investigators carried out this analysis. In addition, the percentage contribution of each retinal layer to the total thickness of the retina was calculated and compared among the different treatment groups (FIG. 2).

### Immunohistochemistry (IHC) and Brightfield Microscopy

Localization of claudin5, occludin and IgG in the cerebral cortex and retina was carried out using immunohistochemistry (IHC) as described previously [16, 20]. Briefly, tissue sections were deparaffinized in xylene and rehydrated in decreasing concentrations of ethanol. The antigenicity of these tissues was enhanced by microwaving in citrate buffer. Sections were treated with 3% H₂O₂ for 10 minutes to quench endogenous peroxidase activity. Tissues were subsequently treated with their appropriate blocking serum for 30 minutes at room temperature and then probed with anti-claudin5 (Invitrogen, Cat #35-2500, dilution 1:150) or anti-occludin (Invitrogen, Cat #71-1500, dilution 1:800) antibodies for 1 hour at room temperature. Extravasated IgG was detected using biotinylated anti-swine IgG antibody (Vector laboratories, Inc., Catalogue # BA-9020; dilution 1:100). The use of occludin antibody required pre-treatment with a protease (Sigma, Catalogue P-5147; 1.5 mg/ml) for 10 minutes at 37° C. At the end of primary antibody step, all tissue sections were thoroughly rinsed in PBS and then probed with biotin-labeled secondary antibodies [anti-mouse IgG (Vector laboratories, Catalogue # PK-6102; Dilution 1:100) for claudin5 and anti-rabbit IgG (Vector laboratories, Inc., Catalogue # PK-6101; Dilution 1:100) for occludin] for 30 minutes at room temperature. Sections were then rinsed in PBS, treated with avidin-peroxidase complex (Vectastain ABC Elite kit, Vector Laboratories, Inc., Catalogue PK-6100), washed with PBS and visualized with 3-3-diaminobenzidine-4-HCL (DAB)/H₂O₂ (Imm-PACT-DAB) (Dako, Code K3468). Nuclei were lightly counterstained with hematoxylin. Lastly, tissues were dehydrated in increasing concentrations of ethanol, cleared in xylene and mounted in Permount (Fisher Scientific, USA). The specificity of the primary antibodies was tested by treating tissue sections with secondary antibody only or blocking sera only as negative controls. Slides were examined and photographed with a Nikon FXA microscope, and digital images were recorded using a Nikon DXM1200F digital camera and analyzed using MATLAB (MathWorks, Mass., USA) and NIS-elements software (Nikon, Melville, N.Y.). During image analysis the identity of the samples was blinded.

### Immunofluorescence Microscopy and Image Analysis of the Retina

Retina sections were deparaffinized using xylene and rehydrated through a graded series of decreasing concentrations of ethanol. Antigen Unmasking Solution (Vector Laboratories) was used according to the manufacturer's instructions. Slides were then placed in 0.5% Triton X-100 in PBS for 5 minutes. After washing briefly with PBS, they were incubated overnight at 4° C. with rabbit anti-GFAP antibody (dilution 1:200, Sigma, Cat # G3893), washed three times with PBS and then incubated with Alexa Fluor 488 goat anti-rabbit secondary antibodies (dilution 1:200, Molecular probes, Life Technology, USA) for 1 hour at room temperature. Sections were thoroughly washed in PBS and then mounted using Vectashield with DAPI (Vector Laboratories, USA). Negative controls were treated with non-immune serum or without primary antibody. Images were taken at 20× magnification on a Nikon Eclipse E800 microscope (Nikon, Melville, N.Y.) equipped with a QImaging Retiga Exi (Qimaging Burnham, Canada) digital camera. Cells positive for GFAP were counted throughout the whole thickness of the retina, and then quantified using ImagePro7.0 (Media Cybernetics, Silver Spring, Md.). Briefly, a 300×300 pixel square area was randomly made in each retinal section in three contiguous locations across the retina and the positive GFAP area was determined per total area using a fixed threshold. For total cell counts, sections were mounted in Vectashield with DAPI (Vector Laboratories). Cell number was determined by counting the DAPI-positive nuclei in a fixed area. The results of these measurements were then averaged for each section. The identity of these samples was blinded during imaging and image analysis.

### Localization and Semi-quantitative Estimation of Claudin5 and Occludin Protein Expression

Claudin5 and occludin protein expression in pig cerebral cortex was evaluated semi-quantitatively using IHC by taking images at five random locations as described previously [16]. Images were analyzed using MATLAB (MathWorks, Mass., USA). During image analysis, optimal conditions were first determined and then standardized for the visualization of claudin5 or occludin in their respective IHC images. These conditions were maintained for all the claudin5 and occludin images, as well as for controls. Using MATLAB, IHC images were first converted into black and white. Sites of claudin5 and occludin localization were rendered white while areas lacking these proteins appeared black. The percentage of total image area occupied by white spots was calculated by MATLAB. Using similar conditions for claudin5 and occludin, the relative expression levels of these were calculated.

### Claudin5 and Occludin Gene Expression

Relative quantification of claudin5 and occludin gene expression was carried out using the reporter dye SYBR green and Applied Biosystems 7500 real time polymerase chain reaction (RT-PCR) system. The primers were generated using Applied Biosystems Primer Express 3.0. Relative quantification of the claudin5 and occludin gene expression was conducted using beta actin (β-actin), a house keeping gene, as a standard. The amplification profiles of all samples were analyzed using Applied Biosystems software. The primers used are as followed: β-actin (forward) 5′-tccagaggcgctcttcca-3′, (reverse) 5′-cgcacttcatgatcgagttga-3′, claudin5 (forward) 5′-cggcgactacgacaagaagaa-3′, (reverse) 5′-gccctccaaagcggagtt-3′, occludin (forward) 5′-tgcacccagcaacgacatata-3′, (reverse) 5′-ggctgagaaagcattggtcga-3′.

### RESULTS

### Chronic DMHC Altered the Thicknesses of the Individual Retinal Layers.

The thickness of the individual layers of the retina was measured in transverse sections stained with Hematoxylin and Eosin using the image analysis software, ImagePro 7.0. In the pig model, chronic DMHC did not cause any significant changes in the total thickness of the retina (FIG. 2A). Despite this, in DMHC animals, it was found a significant decrease in the thicknesses of the ganglion cell layer (GCL), outer nuclear layer (ONL), and inner-segment (IS)/outer-segment (OS) layer compared to controls (FIG. 2B-D) By contrast, the inner plexiform layer (IPL) in DMHC animals showed a significant increase in thickness and was more disorganized compared to controls (FIG. 2E). This could be the reason for similar total retinal thickness among treatment groups in spite of significant reduction in the GCL, ONL and IS/OS. The inner nuclear layer (INL) retained a comparable thickness across all treatment groups (FIG. 2F). Most importantly, Darapladib treatment tended to reverse the effects of DMHC on the GCL, ONL and IS/OS layers as their thickness was comparable to controls (FIG. 2B, 2C-D). Darapladib treatment also lowered IPL thickness in DMHC animals to near control levels (FIG. 2E).

### DMHC Reduced the Number of Cells in the GCL and INL.

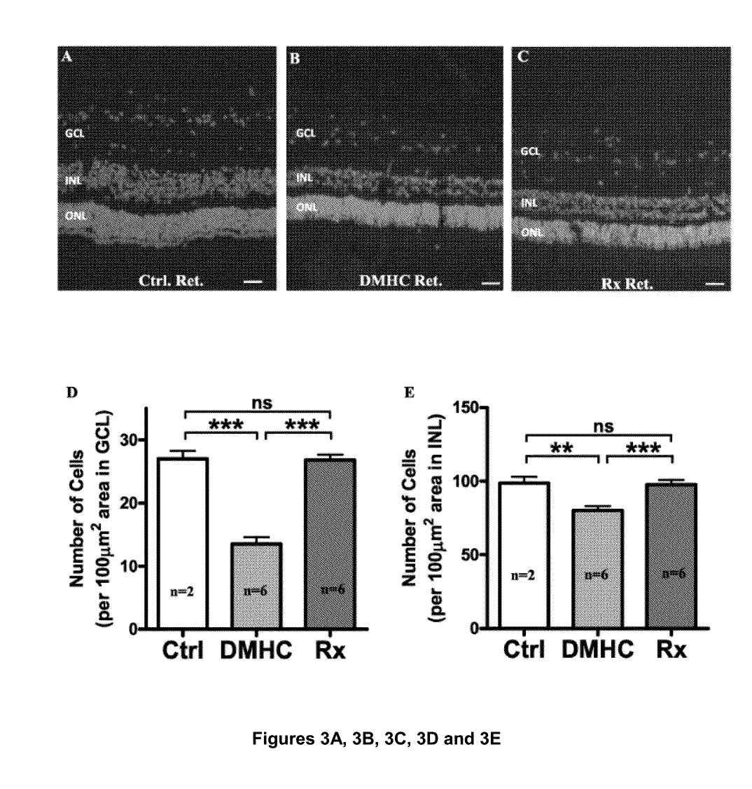

The thickness of any given cellular layer in the retina is directly proportional to the number of cells comprising that layer. To determine the effects of long-term DMHC and DMHC-Darapladib treatment on cell number within the retinal GCL and INL, the relative number of cells in these layers was estimated by counting the nuclei. Histological sections of retinas from all treatment groups were stained with DAPI to reveal nuclei (FIGS. 3A-C). Results showed that DMHC was associated with significant reduction in the numbers of nuclei and therefore cells within the GCL and INL compared to controls (FIGS. 3D-E). On the other hand, treatment with Darapladib inhibited cell loss within these two retinal layers such that measured cell numbers were comparable to controls (FIG. 3D-E).

### IgG Binds Selectively to Ganglion Cells in the GCL.

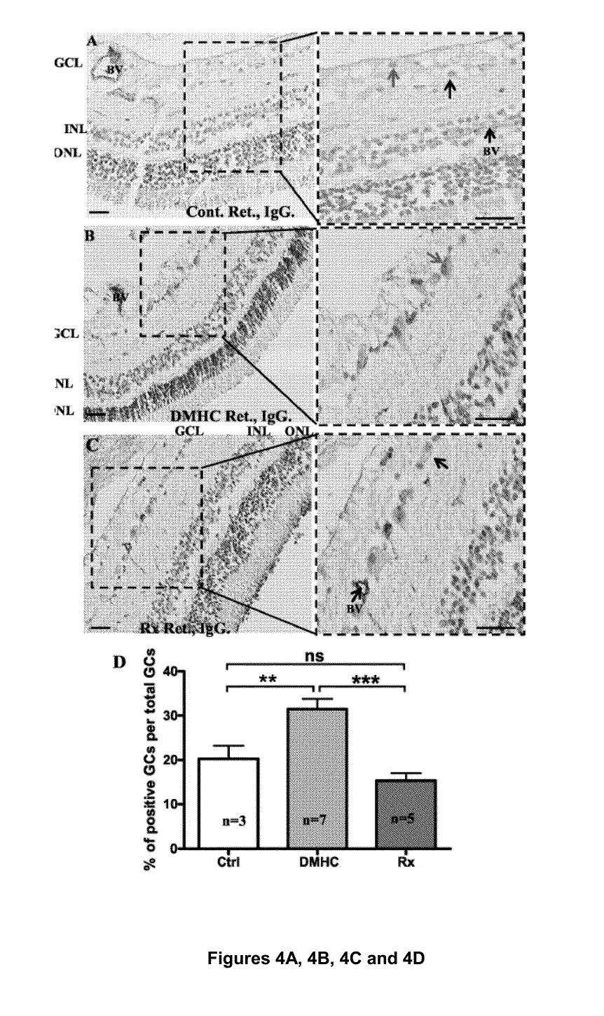

In the present work, to detect extravasated IgG within the retinal tissue as evidence of BRB compromise, retina sections with anti-pig IgG was immunostained. Image analysis revealed an increased presence of IgG-positive ganglion cells in the retina of DMHC pigs. Ganglion cells exhibited both cytoplasmic and cell surface IgG labeling (FIGS. 4A-D). Other retinal layers failed to reveal immuno-labeling with IgG. The percentage of IgG-positive ganglion cells relative to total ganglion cells in the GCL was calculated. Among the treatment groups, DMHC animals had the highest percentage of IgG-positive ganglion cells followed by untreated controls and then the DMHC-Darapladib group (FIG. 4D). Differences between DMHC and the other treatment groups were statistically significant.

### DMHC Triggers Gliosis in the Retina.

Müller cells and astrocytes are the predominant glial cell types within the retina, with Müller cells being more abundant. Under normal conditions in the retina, GFAP is predominantly expressed by astrocytes, while Müller cells rarely express it (Barber et al, 2000; Rungger-Brandle et al, 2000). During DM or in response to retinal injuries, Müller cells become the dominant glial cell type expressing GFAP. Therefore, enhanced expression of GFAP in Müller cells is a reliable telltale signs of ongoing pathological changes within the retina.

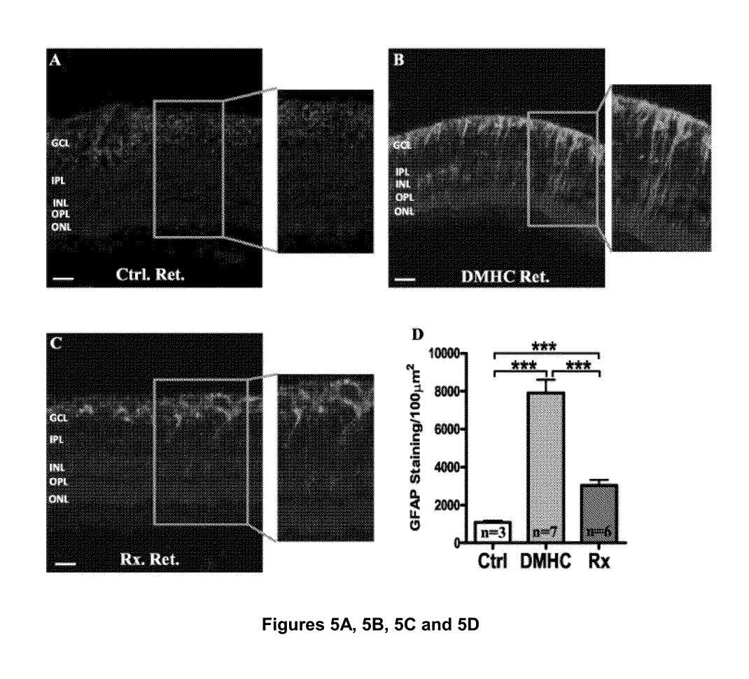

To investigate the level and distribution of GFAP expression in the DMHC pig retina, histological sections from all treatment groups were probed with anti-GFAP antibody. GFAP expression was markedly increased in the DMHC retina compared to controls and DMHC-Darapladib pigs (FIGS. 5A-C). GFAP-positive fluorescence extended from inner to outer limiting membranes in the DMHC group, confirming that GFAP was predominantly being expressed by Müller cells (FIGS. 5A-E). The proximal region of Müller cells localized at the ganglion cell layer demonstrated the most intense GFAP expression in the DMHC group. Here, the distal ends of Müller cells also demonstrated elevated GFAP expression (FIG. 5A). Interestingly, GFAP immunoreactivity was reduced and confined to the GCL in both control and DMHC-Darapladib animals compared to DMHC only (FIGS. 5A-C). The intensity of GFAP immunostaining confirmed the highest level of GFAP expression in DMHC pigs compared to DMHC-Darapladib and control groups (FIG. 5D). Notably, although Darapladib treatment was able to dramatically lower GFAP expression in the retinas of DMHC pigs, it was still significantly higher than in controls (FIG. 5D).

### Chronic DMHC Alters Claudin5 and Occludin Protein Expression in the Cerebral Cortex.

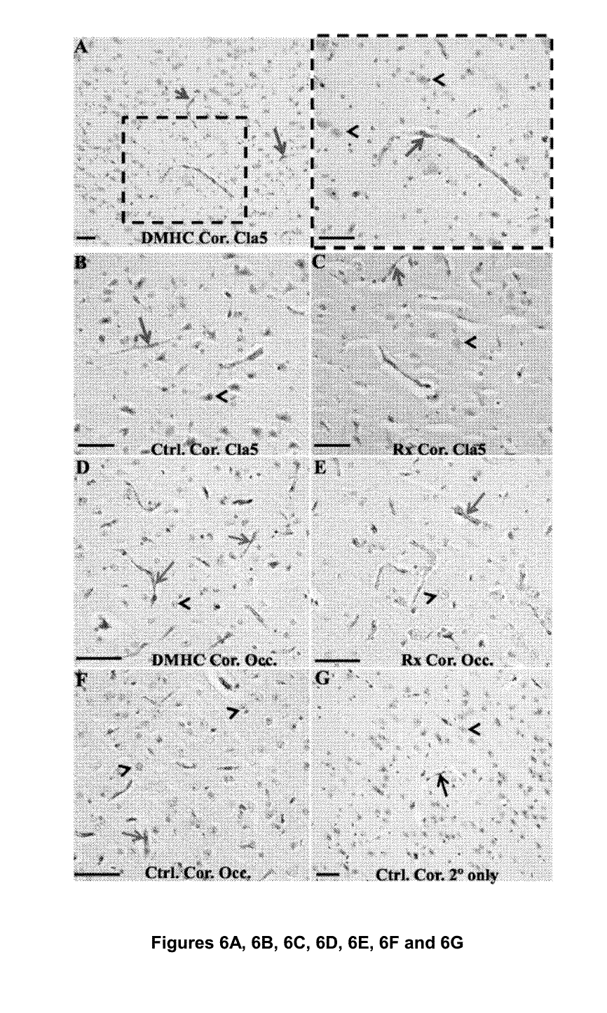

Claudin5 and occludin are expressed in brain vascular endothelial cells (BVECs) that serve as key structural and functional components of the BRB and BBB. The effects of chronic DMHC with or without Darapladib treatment on the localization of claudin5 and occludin in BVECs were investigated. To address this, the cortical blood vessels of this DMHC model were studied. Cerebral cortex instead of the highly laminated retina was chosen for the following two reasons: (1) availability of greater tissue volume for carrying out protein localization studies and (2) the cerebral cortex has an extensive plexus of blood vessels which is readily visualized and thus more amenable to protein localization studies. The expression and localization of claudin5 and occludin were investigated using IHC and image analysis. As expected, these proteins are expressed solely in BVECs and are localized primarily to the tight junctions in all treatment groups (FIGS. 6A-F). Neurons and astrocytes lacked claudin5 and occludin expression (FIG. 6). The specificity of the anti-claudin5 and anti-occludin immunostaining was verified by negative controls that included probing with secondary antibody only (FIG. 6G) or blocking sera only (data not shown).

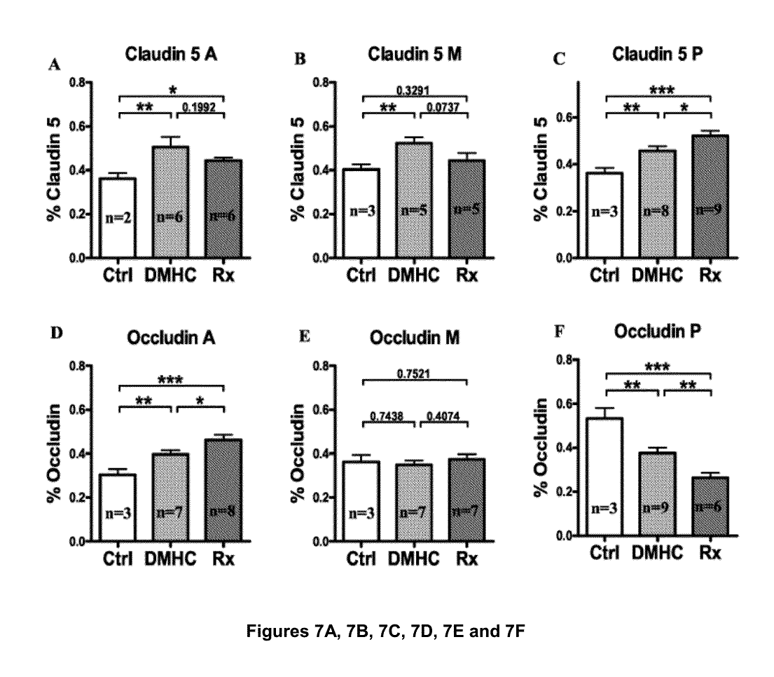

Relative levels of claudin5 and occludin protein expression in the BVECs of the cerebral cortex in DMHC, DMHC-Darapladib and control pigs were measured and compared (FIG. 6). Brains were divided anteroposteriorly into three regions: anterior, middle and posterior. IHC and MATLAB were used to determine claudin5 and occludin protein expression separately in each of the three brain regions in an effort to account for potential region-specific variations in the density and type of vessels. In the anterior brain region, DMHC pigs showed the greatest expression of claudin5, followed by the Darapladib-treated DMHC and age-matched control pigs (FIG. 7A). Claudin5 expression in the anterior cortex of DMHC and DMHC-Darapladib pigs was significantly higher than in controls (FIG. 7A). The DMHC-Darapladib group showed a reduction in claudin5 expression compared to the DMHC group, but this difference did not reach statistical significance. In the middle cortex, claudin5 expression was similar to that of the anterior cortex, being significantly higher in the DMHC group compared to controls (FIG. 7B). The reduction in claudin5 expression in Darapladib-treated DMHC compared to DMHC only animals approached statistical significance. Interestingly, treatment of DMHC animals with Darapladib resulted in a reduced expression of claudin5 to levels not significantly different from controls. Lastly, in posterior cortex, the DMHC-Darapladib group showed the highest expression of claudin5, followed by DMHC and controls, with the variations attaining statistical significance in all treatment groups (FIG. 7C).

The pattern of occludin localization in the cortical vasculature differed from claudin5 for all three brain regions (FIGS. 7 D-F). In the anterior cortex, the DMHC-Darapladib group exhibited the highest level of occludin expression, followed by DMHC and controls (FIG. 7D). The differences among the treatment groups attained statistical significance. In the middle cortex, all treatment groups showed comparable levels of occludin expression (FIG. 7E). In the posterior cortex, the occludin expression pattern was opposite to that of the anterior cortical regions, with the lowest expression measured in the DMHC-Darapladib group and the highest in controls (FIG. 7F). The difference in occludin localization reached statistical significance in the posterior brain region.

### Chronic DMHC Alters Claudin5 and Occludin Gene Expression in the Cerebral Cortex.

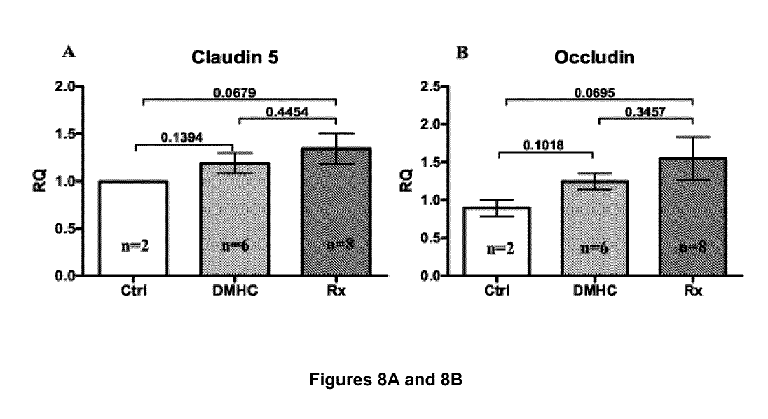

Changes in claudin 5 and occludin gene expression in response to DMHC and Darapladib treatment were assessed in the middle brain region only of the pig cerebral cortex by real-time polymerase chain reaction (RT-PCR) using beta-actin as a reference (FIG. 8). The patterns of claudin5 and occludin gene expression were similar, with the highest claudin5 and occludin levels in the DMHC-Darapladib group, followed by DMHC alone and controls (FIG. 8A-B). The difference in both claudin5 and occludin gene expression between the DMHC-Darapladib and control groups approached statistical significance.

These two studies strongly suggest that the influx of IgG into the brain and retina enables its binding to cell surfaces and promotes chronic endocytosis which, in the case of cortical pyramidal neurons, has been linked to intracellular Aβ42 deposition, a feature presumably contributing to Alzheimer's disease pathogenesis and progression. In a similar manner, this binding of IgG to ganglion cells may, through the capacity of IgG for crosslinking, interfere with the normal function of their cytoplasmic processes. The data suggests direct link between influx of plasma components into the retina with observed changes in the thickness and cell loss within individual retinal layers. The fact that Darapladib treatment not only curtailed plasma and IgG influx into retinal layers but also stabilized the thickness of retinal layers and cell number further supports the likely link between plasma influx into the brain and retina and degenerative changes.

Increased GFAP expression is widely used as a reliable biomarker of glial cell activation, which usually occurs in response to injury resulting from trauma, disease, inflammation or chemical insults. In the present work, chronic DMHC was found to trigger gliosis in the retina as evidenced by increased expression of GFAP by Müller cells. Two types of glial cells populate the mammalian retina: Müller cells and astrocytes. The cell bodies of Müller cells are positioned within the GCL, and their cytoplasmic processes extend to the inner and outer limiting membranes of the retina. Astrocytes are less abundant in the retina and form a single layer in the GCL with close proximity to the inner limiting membrane. Under normal, healthy conditions in the retina, GFAP is predominantly expressed by astrocytes, while Müller cells rarely express it. However, under conditions of DM or retinal injury as shown here, Müller cells become the dominant glial cell type expressing GFAP. This change suggests ongoing oxidative stress as well as inflammatory and other degenerative changes in the retina possibly as a result of DMHC. Notably, in the retinas of Darapladib-treated DMHC pigs, GFAP expression was significantly lowered and was limited to GCL. As such, for the first time, beneficial effects of Darapladib in inhibiting or alleviating DMHC-associated pathological changes in the retina layers and sub-layer were revealed.

It will be appreciated by persons skilled in the art that the present invention is not limited to what has been particularly shown and described. Rather, the scope of the present invention is defined by the claims which follow. It should further be understood that the above description is only representative of illustrative examples of embodiments. The description has not attempted to exhaustively enumerate all possible variations. The alternate embodiments may not have been presented for a specific portion of the invention, and may result from a different combination of described portions, or that other un-described alternate embodiments may be available for a portion, is not to be considered a disclaimer of those alternate embodiments. It will be appreciated that many of those un-described embodiments are within the literal scope of the following claims, and others are equivalent.

1. A method of treating or preventing an eye disease in a subject in need thereof, comprising:
(a) identifying a subject with an eye disease or at risk of having the eye disease or disorder, comprising measuring the thickness of one or more retinal layers selected from ganglion cell layer (GCL), outer nuclear layer (ONL), and inner-segment (IS); and
(b) administering to said subject in need thereof a pharmaceutical composition comprising an effective amount of an agent for inhibiting the activity or expression of the Lp-PLA₂ protein.

2. The method of claim 1, further comprising measuring the thickness of inner plexiform layer (IPL).

3. The method of claim 1, wherein step (a) comprises measuring the thickness of GCL, ONL, or both.

4. The method of claim 1, wherein the thickness is measured with optical coherence tomography.

5. The method of claim 1, wherein said eye disorder is characterized by one or more disease states selected from vitreous hemorrhage, progressive fibrovascular proliferation, retinal detachment, rubeosis iridis, and neovascular glaucoma.

6. The method of claim 1, wherein said eye disease is selected from the group consisting of central retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema.

7. The method of claim 1, wherein said eye disease is associated with age-related macular degeneration (AMD), diabetic retinopathy (DR), or both.

8. The method of claim 1, wherein the agent is selected from the group consisting of darapladib and rilapladib.

9. The method of claim 1, further comprising measuring the one or more retinal layers after the initial administration of the agent and adjusting the dosage of the agent accordingly.

10. The method of claim 1, further comprising administering to the subject a second agent selected from the group consisting of Corticosteroids, VEGF inhibitors, PKC inhibitors, and growth hormone inhibitors.

11. A method of detecting the extent of a neurodegenerative disease or the risk of the neurodegenerative disease in a subject comprising measuring the thickness of one or more retinal layers selected from ganglion cell layer (GCL), outer nuclear layer (ONL), and inner-segment (IS), wherein an increase in the thickness of one or more retinal layers is indicative of the extent of the neurodegenerative disease or the risk level of said subject developing an eye disease.

12. The method of claim 11, further comprising:
comparing the measured thickness with a reference; and
generating a report specifying that the extent of the neurodegenerative disease or the risk of the neurodegenerative disease in the subject.

13. The method of claim 12, wherein the neurodegenerative disease is selected from the group consisting of Alzheimer's disease, Multiple Sclerosis, Parkinson's disease, frontotemporal dementia, vascular cognitive impairment (formerly vascular dementia), and dementia with Lewy body.

14. A method of monitoring the treatment response in a subject being treated for a neurodegenerative disease or an eye disease, comprising measuring the thickness of one or more retinal layers selected from ganglion cell layer (GCL), outer nuclear layer (ONL), and inner-segment (IS) after administration of an agent to the subject, wherein a reduction in the thickness of one or more said retinal layers is indicative a good treatment response and a lack of reduction in the thickness of one or more said retinal layers is indicative a poor treatment response.

15. The method of claim 14, further comprising comparing the thickness of the one or more retinal layers with a reference and generating a report, wherein the reference is the thickness of the one or more retinal layers prior to administration of the agent.

16. The method of claim 14, further comprising comparing the thickness of the one or more retinal layers with a reference and generating a report, wherein the reference is the normal thickness of the one or more retinal layers.

17. The method of claim 14, wherein said neurodegenerative disease is selected from the group consisting of Alzheimer's disease, Multiple Sclerosis, Parkinson's disease, frontotemporal dementia, vascular cognitive impairment (formerly vascular dementia), and dementia with Lewy body.

18. The method of claim 14, wherein the eye disease is selected from eye disease is selected from the group consisting of retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema.

19. A system for detecting or diagnosing an eye disease or a neurodegenerative disease in a subject in need, comprising
(a) a detection unit for measuring the thickness of a retinal layer of said subject; and
(b) a database unit for providing the relationship between the thickness of a retinal layer and the risk or extent of the eye disease or a neurodegenerative disease.

20. The system of claim 19, wherein the detection unit comprises optical coherence tomography.

21. The system of claim 19, wherein the eye disease is selected from the group consisting of retinal vein occlusion, branched retinal vein occlusion, Irvine-Gass syndrome (post cataract and post-surgical), retinitis pigmentosa, pars planitis, birdshot retinochoroidopathy, epiretinal membrane, choroidal tumors, cystic macular edema, parafoveal telengiectasis, tractional maculopathies, vitreomacular traction syndromes, retinal detachment, neuroretinitis, and idiopathic macular edema.

22. The system of claim 19, wherein the neurodegenerative disease is selected from the group consisting of Alzheimer's disease, Multiple Sclerosis, Parkinson's disease, frontotemporal dementia, vascular cognitive impairment (formerly vascular dementia), and dementia with Lewy body.
